(19) United States
(12) Patent Application Publication
(10) Pub. No.: US 2023/0343417 A1
(43) Pub. Date: Oct. 26, 2023

(54) INFORMATION ANALYSIS APPARATUS, INFORMATION ANALYSIS METHOD, AND INFORMATION ANALYSIS PROGRAM

(71) Applicant: FRONTEO, Inc., Tokyo (JP)

(72) Inventor: Hiroyoshi TOYOSHIBA, Tokyo (JP)

(21) Appl. No.: 18/255,595

(22) PCT Filed: Mar. 3, 2021
(86) PCT No.: PCT/JP2021/008137

§ 371 Date: Jun. 2, 2023

(51) Int. Cl. G16B 40/00; G16B 45/00; G16B 50/00

(52) U.S. Cl. CPC G16B 45/00; G16B 40/00; G16B 50/00

(57) ABSTRACT

A 2D map generation unit 3 that generates a 2D map in which positions corresponding to a plurality of feature vectors are visualized on a 2D plane based on a plurality of pieces of 2D coordinate information obtained by performing dimension compression on a plurality of word feature vectors specified for each of a plurality of words included in a plurality of texts, and a pathway generation unit 2 that uses a similarity of a plurality of word feature vectors or uses a position or a range designated in the 2D map to specify a plurality of molecules as words, and uses a knowledge database showing an intermolecular connection relationship for the specified plurality of molecules to generate a pathway representing an intermolecular interaction as a route map are included, and an environment for information analysis using a 2D map and a pathway together is provided.

### TECHNICAL FIELD

The present invention relates to an information analysis apparatus, an information analysis method, and an information analysis program, and more particularly to a technology of expressing a feature of information by a feature vector and performing information analysis using the feature vector.

### BACKGROUND ART

Conventionally, there has been known a technology of expressing a feature of information by a feature vector and performing information analysis using the feature vector (for example, see Patent Documents 1 to 3).

Patent Document 1 discloses method of predicting a protein-protein interaction having potential as a drug target by performing supervised machine learning with predetermined attributes related to proteins as feature vectors. In a prediction system described in Patent Document 1, machine learning is performed using an attribute of a biological function of each protein as one predetermined attribute related to the protein. Patent Document 1 discloses that the number of pathways containing each protein is used as one attribute of the biological function of each protein.

The pathway is a route map which represents a molecule of a gene, a protein, etc. using a symbol such as a circle or a square, and is expressed by connecting symbols with arrows that represent intermolecular interactions. Such visualization of the intermolecular interaction allows easier understanding of life phenomena such that it is possible to investigate a path containing a gene group whose expression level has changed. For example, a pathway is widely used in a field of disease treatment or drug discovery.

There are two types of pathways, one created manually and the other created using a computer. The former pathway is created mainly by researchers reading biochemical or medical literature and drawing content described therein as a text as a route map of the pathway. The latter pathway is created, for example, by reading a text described in a literature as text data and depicting described content whose meaning is interpreted by natural language processing as a route map.

However, a conventional pathway created manually is merely obtained by a creator depicting a known intermolecular interaction understood from description of a literature as a pathway. Therefore, the pathway that can be manually created is limited to a range of described content of the literature read by the creator. A conventional pathway created by a computer is also basically similar thereto, and the pathway that can be created is limited to a range of described content of a literature read by the computer as text data. More literature can be read in the case of a computer than in the case of a human, and a width of a pathway that can be created increases accordingly. However, a known intermolecular interaction described in the literature is merely depicted.

Patent Document 2 and 3 disclose technology for displaying a 2D map in which a plurality of search targets is plotted on a 2D plane based on a feature vector generated from a search target, extracting search targets corresponding to plots included in a region designated by a user operation, and displaying a list of the extracted search targets.

A document search apparatus described in Patent Document 2 displays a map in which a plurality of documents is plotted on a 2D plane based on a document vector. Then, when a user designates a desired region on a 2D map in which a plot is positioned according to a degree of relevance between documents in this way, query vectors of a plurality of documents contained in the designated region are synthesized, a document vector in an information database is compared with a synthetic query vector, and documents corresponding to document vectors close to the synthetic query vector are extracted and displayed in a list.

In the document search apparatus described in Patent Document 2, a 2D map generator reads from the information database a document vector corresponding to a document extracted based on a search keyword entered by the user, and calculates a similarity between respective documents. The 2D map generator reduces the dimension of a multidimensional document vector to obtain a 2D document vector and performs conversion into an x-coordinate and a y-coordinate so that similar documents are placed closer together on the 2D map based on the similarity between the respective document vectors. The 2D map generator creates a coordinate list of the x-coordinate and the y-coordinate of each document, and creates a 2D map based on the coordinate list.

An information search apparatus described in Patent Document 3 generates and displays a 2D map illustrating respective information items corresponding to respective positions in an array so that similar information items are mapped to close positions based on a similarity of information items from a set of the information items. Further, when the user performs an operation to define an arbitrary boundary region on the 2D map, by specifying an information item which is present as information indicating a position in the defined boundary region and corresponds to a position in the array as an item corresponding to a search query, related search is performed for the boundary region, and a list of information items specified as a result of the related search is displayed.

In the information search apparatus described in Patent Document 3, for example, the information item is a document. The information search apparatus generates a multidimensional feature vector based on an abstract expression representing a frequency of a term used in a document (for example, a term frequency histogram composed by counting the number of times a word in a dictionary appears in an individual document). Then, after reducing the dimension of the feature vector, a semantic map is created by projecting the feature vector onto a 2D self-organizing map. By assigning the feature vector for each document to the map, a map position according to an x-coordinate and a y-coordinate is generated for each document, and a relationship between documents can be visualized according to a position thereof.
Patent Document 1: JP-A-2010-165230
Patent Document 2: Japanese Patent No. 5,159,772
Patent Document 3: Japanese Patent No. 4,540,970

A pathway created by a conventional method can be used when researching and developing a new drug or a new treatment effective against a disease for which an effective treatment or drug has not been established, etc. However, since the conventional pathway merely depicts the known intermolecular interaction described in the literature, etc., it is difficult to obtain knowledge beyond human intelligence from the pathway. In particular, for a newly developed disease of an unknown property or a pathogen of unknown identity, etc., there is a problem that it is difficult to obtain, from the conventional pathway, knowledge such as a type of molecule involved or a type of existing drug which is effective as a research target.

Note that a technology described in Patent Document 1 does not create a pathway using machine learning, but performs machine learning using attributes related to a plurality of pathways that have been previously created. Patent Document 1 fails to disclose a method of creating a pathway.

In the technologies described in Patent Documents 2 and 3, a 2D map plotted so that similar documents are disposed close to each other is displayed, and a document located within a designated region on the 2D map is extracted. For this reason, it is possible to efficiently extract a plurality of similar documents. However, for example, in researching and developing a new drug or a new treatment method that is effective for a disease for which an effective treatment method or drug has not been established, etc., for example, even when a plurality of similar documents regarding the disease can be efficiently extracted, it is necessary for a human to read and understand content described in the plurality of documents. However, it is difficult to obtain knowledge beyond the human knowledge that is not described in the document by simply reading the document.

The invention has been made in view of such circumstances, and an object of the invention is to provide an information analysis method useful for obtaining new knowledge beyond a range of known knowledge described in literature, etc.

### SOLUTION TO PROBLEM

To solve the above-mentioned problem, the invention uses a plurality of feature vectors including a plurality of word feature vectors specified for each of a plurality of words included in a plurality of texts to generate a 2D map in which positions corresponding to the plurality of feature vectors are visualized on a 2D plane based on a plurality of pieces of 2D coordinate information obtained by performing dimension compression on each of the plurality of feature vectors. In addition, the invention uses a similarity of a plurality of word feature vectors or uses a position or a range designated in a 2D map to specify a plurality of molecules as words, and uses a knowledge database showing an intermolecular connection relationship for the specified plurality of molecules to generate a pathway representing an intermolecular interaction as a route map.

### ADVANTAGEOUS EFFECTS OF THE INVENTION

According to the invention configured as described above, a 2D map and a pathway are generated based on a plurality of feature vectors obtained from a plurality of texts. In a 2D map generated in the invention, a coordinate position is determined according to a property of information of each of a plurality of feature vectors, feature vectors having similar properties are disposed at positions close to each other, and feature vectors having dissimilar properties are disposed at positions far apart from each other. Further, a pathway generated in the invention is not merely obtained by visualizing known content described in a literature by a human or a computer as a route map, and is generated and visualized using a property of information of each of a plurality of feature vectors. A user can perform information analysis useful for obtaining new knowledge beyond a range of known knowledge described in a literature, etc. using a 2D map and a pathway having such properties together.

### BRIEF DESCRIPTION OF THE DRAWINGS

FIG. 1 is a diagram illustrating an overall configuration example of an information providing system including an information analysis apparatus according to a first embodiment.

FIG. 2 is a block diagram illustrating a functional configuration example of a server apparatus (information analysis apparatus) according to the first embodiment.

FIG. 3 is a diagram illustrating an example of a pathway displayed on a display apparatus in the first embodiment.

FIG. 4 is a diagram illustrating an example of a 2D map displayed on the display apparatus in the first embodiment.

FIG. 5 is a block diagram illustrating a functional configuration example of a feature vector computation apparatus.

FIG. 6 is a diagram illustrating an example of a disease feature vector and a molecule feature vector.

FIG. 7 is a block diagram illustrating a specific functional configuration example of a pathway generation unit according to the first embodiment.

FIG. 8 is a block diagram illustrating a specific functional configuration example of a 2D map generation unit according to the first embodiment.

FIG. 9 is a diagram illustrating an example of a linked display of the pathway and the 2D map in the first embodiment.

FIG. 10 is a diagram illustrating another example of the linked display of the pathway and the 2D map in the first embodiment.

FIG. 11 is a block diagram illustrating a functional configuration example of a server apparatus (information analysis apparatus) according to a second embodiment.

FIG. 12 is a diagram illustrating an example of a text feature vector.

FIG. 13 is a diagram illustrating an example of a plurality of types of 2D maps displayed on a display apparatus in the second embodiment.

FIG. 14 is a block diagram illustrating a specific functional configuration example of a pathway generation unit according to the second embodiment.

FIG. 15 is a diagram illustrating an example of a linked display of a plurality of types of 2D maps in the second embodiment.

FIG. 16 is a diagram illustrating another example of the linked display of the plurality of types of 2D maps in the second embodiment.

### MODE FOR CARRYING OUT THE INVENTION

### First Embodiment

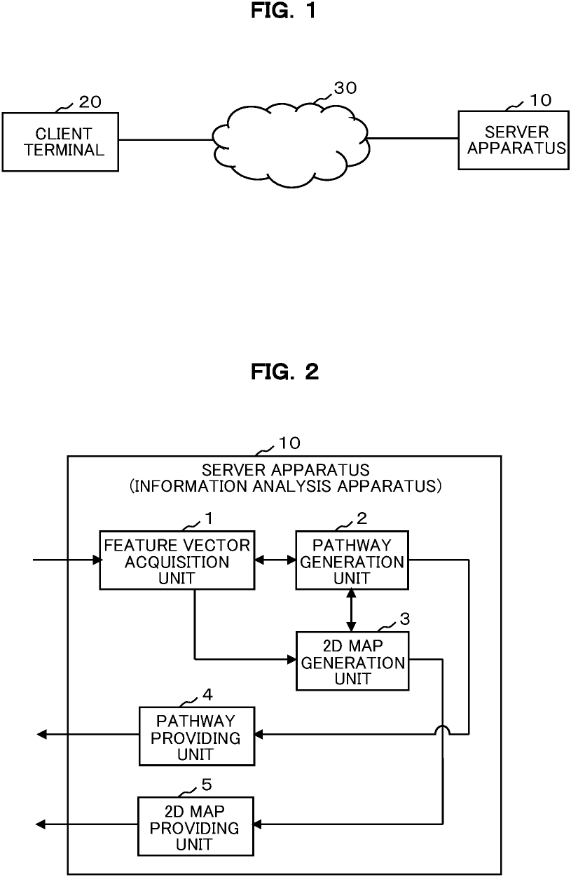

Hereinafter, a first embodiment of the invention will be described with reference to the drawings. FIG. 1 is a diagram illustrating an overall configuration example of an information providing system including an information analysis apparatus according to the first embodiment. As illustrated in FIG. 1, the information providing system of the present embodiment includes a server apparatus 10 and a client terminal 20, and the server apparatus 10 and the client terminal 20 are connected by a communication network 30 such as the Internet. The server apparatus 10 includes the information analysis apparatus of the first embodiment.

In the information providing system of the present embodiment, when the server apparatus 10 is requested to provide a pathway from the client terminal 20, the server apparatus 10 generates a pathway representing as a route map an intermolecular interaction, and provides the generated pathway to the client terminal 20. The client terminal 20 displays the pathway provided from the server apparatus 10 on the display apparatus. Further, in the information providing system of the present embodiment, when the server apparatus 10 is requested to provide a 2D map from the client terminal 20, the server apparatus 10 generates a 2D map in which a plurality of molecules is visualized at positions on a 2D plane, and provides the generated 2D map to the client terminal 20. The client terminal 20 displays the 2D map provided by the server apparatus 10 on the display apparatus. The client terminal 20 can perform such a process using a web browser, for example.

In the first embodiment, either a pathway or a 2D map may be independently displayed on the client terminal 20, or both the pathway and the 2D may be displayed on the client terminal 20 at the same time. Alternatively, after generating a pathway and displaying the pathway on the client terminal 20, information about a plurality of molecules included in the pathway may be used to generate a 2D map, and the 2D map may be displayed on the client terminal 20.

FIG. 2 is a block diagram illustrating a functional configuration example of the server apparatus 10 (information analysis apparatus) according to the first embodiment. As illustrated in FIG. 2, the server apparatus 10 according to the first embodiment includes a feature vector acquisition unit 1, a pathway generation unit 2, a 2D map generation unit 3, a pathway providing unit 4, and a 2D map providing unit 5 as functional configurations.

Each of the functional blocks 1 to 5 can be configured by any of hardware, digital signal processor (DSP), and software. For example, when configured by software, each of the functional blocks 1 to 5 is actually configured to include a CPU, a RAM, a ROM, etc. of a computer, and is implemented by operating an information analysis program stored in a storage medium such as a RAM, a ROM, a hard disk, or a semiconductor memory.

The feature vector acquisition unit 1 acquires a plurality of feature vectors including a plurality of word feature vectors specified for each of a plurality of words included in a plurality of texts. For example, the feature vector acquisition unit 1 acquires a word feature vector (hereinafter referred to as a molecule feature vector) specified for a molecule name and a word feature vector (hereinafter referred to as a disease feature vector) specified for a disease name.

The molecule feature vector used here is data representing a feature (feature that can identify a molecule) of a molecule of a protein, a gene, etc. as a combination of values of a plurality of elements. In the present embodiment, as an example, a vector representing a text to which a molecule name included as a word in a plurality of texts contributes and a degree at which the molecule name contributes to the text is used as a molecule feature vector.

Similarly, the disease feature vector is data representing features of the disease (features that can identify the disease) as a combination of values of a plurality of elements. In the present embodiment, as an example, a vector representing a text to which a disease name included as a word in a plurality of texts contributes and a degree at which the disease name contributes to the text is used as a disease feature vector.

The text to be target in the present embodiment may include one sentence (a unit separated by a period) (one statement), or include a plurality of sentences. A text including a plurality of sentences may be a part or all of a text contained in one document. The target text is not limited to a description related to a molecule or disease, and may include a description of various other themes.

While a molecule name or disease name as a word tends to be used in a text describing a molecule or disease, the molecule name or disease name tends not to be used in a text unrelated to the molecule or disease. In addition, among texts describing a molecule or disease, a text containing a certain molecule name or disease name as a word is a text describing the molecule or disease, and it is highly possible that the molecule name or disease name is not included in a text describing another type of molecule or disease. That is, a text containing a molecule name or disease name as a word tends to differ depending on the type of molecule or disease which is a theme of the text. Therefore, a vector representing a text to which a molecule name or disease name contributes and a degree at which the molecule name or disease name contributes to the text may be used as a feature vector that can identify a molecule or disease.

In the first embodiment, the feature vector acquisition unit 1 acquires a disease feature vector which is a word feature vector specified for a name of a disease to be analyzed. Further, if necessary, the feature vector acquisition unit 1 acquires a molecule feature vector of a plurality of molecules presumed to be related to the disease to be analyzed based on a similarity between the acquired disease feature vector and molecule feature vectors.

Here, if a pathway will be generated in a state in which a 2D map is not generated, the feature vector acquisition unit 1 acquires a disease feature vector corresponding to a disease name (disease name designated by a user as an analysis target) included in a pathway acquisition request received from the client terminal 20. Further, when a 2D map is generated in a state in which a pathway is not generated, the feature vector acquisition unit 1 acquires a disease feature vector corresponding to a disease name included in a 2D map acquisition request received from the client terminal 20.

For example, a disease to be analyzed is designated by a user of the client terminal 20 operating a keyboard or a touch panel and inputting a name of the disease to be analyzed. Note that the disease to be analyzed may be designated by the user of the client terminal 20 operating a mouse or the touch panel and selecting the name of the disease to be analyzed from a display list. The client terminal 20 transmits the pathway acquisition request or the 2D map acquisition request including the disease name designated by the user as described above to the server apparatus 10. In response to this acquisition request, the feature vector acquisition unit 1 of the server apparatus 10 acquires a disease feature vector corresponding to the disease name included in the acquisition request.

If the 2D map will be generated in the state in which the pathway is not generated, the feature vector acquisition unit 1 acquires the disease feature vector as described above, and then further acquires a molecule feature vector of a plurality of molecules presumed to be related to the disease to be analyzed based on a similarity between the acquired disease feature vector and molecule feature vectors.

The similarity between the disease feature vector and the molecule feature vector can be evaluated by various methods. For example, it is possible to apply a method of extracting a feature quantity using a predetermined function for each of the disease feature vector and the molecule feature vector and evaluating a similarity of the feature quantity. Alternatively, it is possible to use a Euclidean distance or cosine similarity between each constituent element of the disease feature vector and each constituent element of the molecule feature vector, or it is possible to use an edit distance. For example, the feature vector acquisition unit 1 acquires a plurality of molecule feature vectors whose evaluation values (degrees of similarity) related to these similarities are equal to or higher than a predetermined value.

Here, for example, the feature vector acquisition unit 1 includes a database (not illustrated) associating and storing a word name (a disease name or a molecule name) with a word feature vector (a disease feature vector or a molecule feature vector) corresponding thereto, and acquires a word feature vector by reading a necessary disease feature vector from the database in response to a pathway or 2D map acquisition request sent from the client terminal 20 and reading a molecule feature vector of a plurality of molecules presumed to be related to a disease to be analyzed from the database as necessary. The word feature vector stored in the database is computed in advance by a feature vector computation apparatus described later.

As another example, when a pathway or 2D map acquisition request is received from the client terminal 20, the feature vector acquisition unit 1 may compute a necessary disease feature vector and molecule feature vector in real time in response to the acquisition request. That is, the feature vector acquisition unit 1 may have a function of the feature vector computation apparatus described later, and the disease feature vector and the molecule feature vector may be acquired by executing the function of the feature vector computation apparatus.

Note that when a 2D map is generated from a previously generated pathway, the feature vector acquisition unit 1 may acquire a text feature vector of a plurality of molecules included in the pathway.

The pathway generation unit 2 specifies a plurality of molecules as words by utilizing a similarity of a plurality of word feature vectors acquired by the feature vector acquisition unit 1, and generates a pathway representing an intermolecular interaction as a route map using a knowledge database showing an intermolecular connection relationship for the specified plurality of molecules. Specifically, the pathway generation unit 2 specifies a plurality of molecules presumed to be related to a disease to be analyzed based on a similarity between the disease feature vector and the molecule feature vector acquired by the feature vector acquisition unit 1, and generates a pathway using a knowledge database showing an intermolecular connection relationship for the specified plurality of molecules. A specific example of generation content of this pathway will be described later.

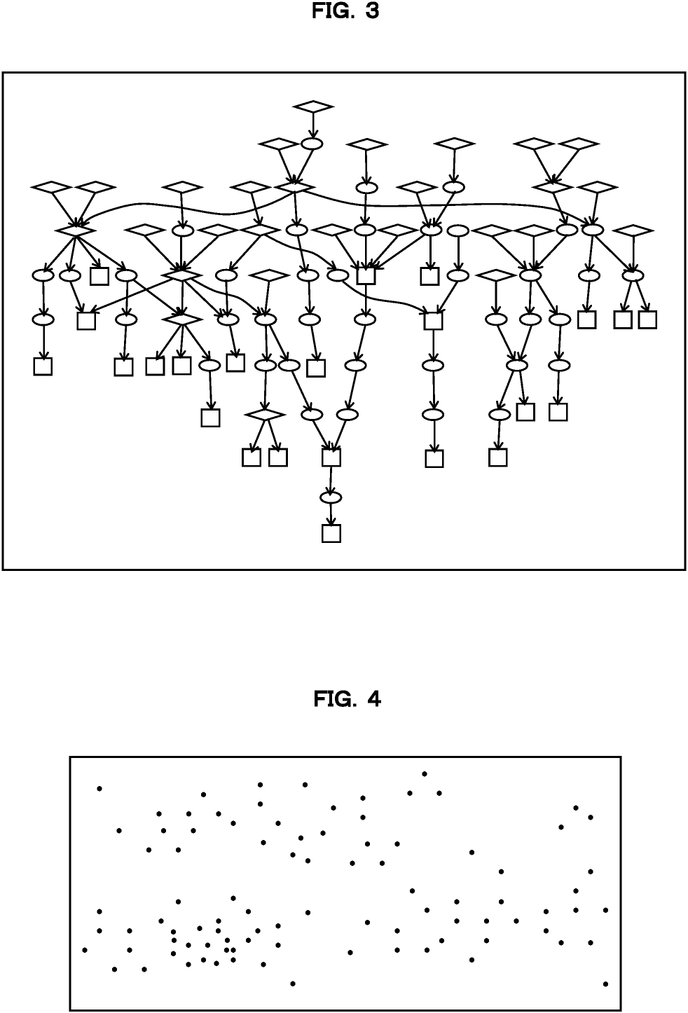

The pathway provision unit 4 provides the client terminal 20 with the pathway data generated by the pathway generation unit 2. The client terminal 20 acquires the pathway data provided from the server apparatus 10, and displays the pathway on the display apparatus. FIG. 3 is a diagram illustrating an example of a pathway displayed on the display apparatus of the client terminal 20. Note that details thereof will be described later.

The 2D map generation unit 3 generates a plurality of pieces of 2D coordinate information by performing dimension compression on the plurality of molecule feature vectors acquired by the feature vector acquisition unit 1, and generates a 2D map in which positions corresponding to the plurality of molecule feature vectors are visualized on a 2D plane based on the generated plurality of pieces of 2D coordinate information. A specific example of generation content of this 2D map will be described later.

The 2D map providing unit 5 provides the client terminal 20 with data of the 2D map generated by the 2D map generation unit 3. The client terminal 20 acquires the data of the 2D map provided by the server apparatus 10 and displays the 2D map on the display apparatus. FIG. 4 is a diagram illustrating an example of the 2D map displayed on the display apparatus of the client terminal 20. In the first embodiment, the number of molecules plotted on the 2D map is the same as the number of molecules displayed as symbols on the pathway.

Next, a specific description will be given of an example of a computation method for a word feature vector (a disease feature vector and a molecule feature vector).

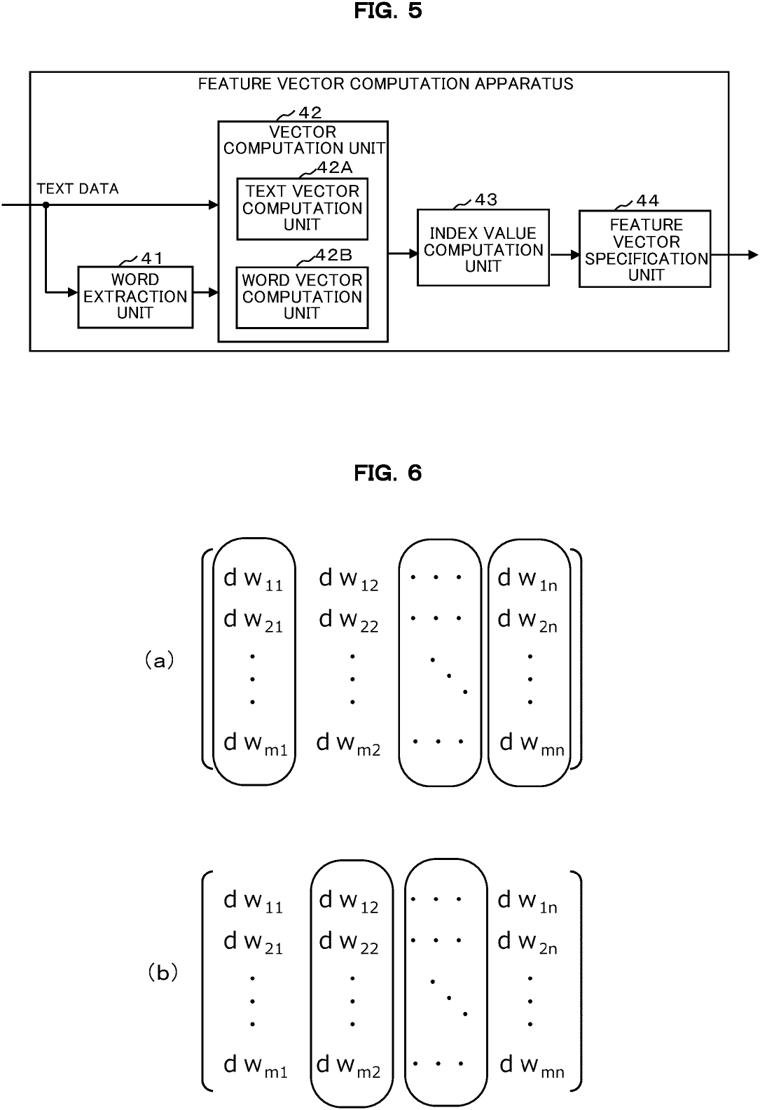

FIG. 5 is a block diagram illustrating a functional configuration example of the feature vector computation apparatus. The feature vector computation apparatus illustrated in FIG. 5 inputs text data related to a text, and computes and outputs a word feature vector reflecting a relationship between the text and a word contained therein. When the feature vector acquisition unit 1 has a function of this feature vector computation apparatus and computes the word feature vector in real time, the server apparatus 10 stores text data related to a plurality of texts, and the feature vector acquisition unit 1 computes the word feature vector using the text data.

As illustrated in FIG. 5, the feature vector computation apparatus includes a word extraction unit 41, a vector computation unit 42, an index value computation unit 43, and a feature vector specification unit 44 as functional configurations thereof. The vector computation unit 42 includes a text vector computation unit 42A and a word vector computation unit 42B as more specific functional configurations.

Each of the functional blocks 41 to 44can be configured by any of hardware, a DSP, and software. For example, in the case of being configured by software, each of the functional blocks 41 to 44 actually includes a CPU, a RAM, a ROM, etc. of a computer, and is implemented by operation of a program stored in a recording medium such as a RAM, a ROM, a hard disk, or a semiconductor memory.

The word extraction unit 41 analyzes m texts (m is an arbitrary integer of 2 or more) and extracts n words (n is an arbitrary integer of 2 or more) from the m texts. As a method of analyzing texts, for example, a known morphological analysis can be used. The word extraction unit 41 may extract morphemes of all parts of speech divided by the morphological analysis as words, or may extract only morphemes of a specific part of speech as words.

Alternatively, only words of a disease name and a molecule name designated in advance may be extracted.

Note that the same word may be included in the m texts a plurality of times. In this case, the word extraction unit 41 does not extract the plurality of the same words, and extracts only one. That is, the n words extracted by the word extraction unit 41 refer to n types of words.

The vector computation unit 42 computes m text vectors and n word vectors from the m texts and the n words. Here, the text vector computation unit 42A converts each of the m texts to be analyzed by the word extraction unit 41 into a q-dimensional vector (q is an arbitrary integer of 2 or more) according to a predetermined rule, thereby computing the m text vectors including q axis components. In addition, the word vector computation unit 42B converts each of the n words extracted by the word extraction unit 41 into a q-dimensional vector according to a predetermined rule, thereby computing the n word vectors including q axis components.

In the present embodiment, as an example, a text vector and a word vector are computed as follows. Now, a set S = <d ∈ D, w ∈ W> including the m texts and the n words is considered. Here, a text vector dᵢ→ and a word vector wⱼ→ (hereinafter, the symbol “→” indicates a vector) are associated with each text dᵢ (i = 1, 2, ..., m) and each word wⱼ (j = 1, 2, ..., n), respectively. Then, a probability P (wⱼ | dᵢ) shown in the following Equation (1) is calculated with respect to an arbitrary word wⱼ and an arbitrary text dᵢ.

### Equation 1

$$\text{P} \left({\text{w}}_{\text{j}} \left(\right| {\text{d}}_{\text{i}}\right) = \frac{exp \left({\vec{\text{w}}}_{\text{j}} ⋅ {\vec{\text{d}}}_{\text{i}}\right)}{{\sum }_{\text{k} = 1}^{\text{n}} exp \left({\vec{\text{w}}}_{\text{k}} ⋅ {\vec{\text{d}}}_{\text{i}}\right)} \qquad \text{(1)}$$

Note that the probability P(wⱼ|dᵢ) is a value that can be computed in accordance with a probability p disclosed in, a follow known document. “‘Distributed Representations of Sentences and Documents’ by Quoc Le and Tomas Mikolov, Google Inc; Proceedings of the 31st International Conference on Machine Learning Held in Bejing, China on 22-24 Jun. 2014” This known document states that, for example, when there are three words “the”, “cat”, and “sat”, “on” is predicted as a fourth word, and a computation formula of the prediction probability p is described.

The probability p(wt|wt - k, ..., wt + k) described in the known document is a correct answer probability when another word wt is predicted from a plurality of words wt - k, ..., wt + k. Meanwhile, the probability P (wⱼ | dᵢ) shown in Equation (1) used in the present embodiment represents a correct answer probability that one word wⱼ of n words is predicted from one text dᵢ of m texts. Predicting one word wⱼ from one text dᵢ means that, specifically, when a certain text dᵢ appears, a possibility of including the word wⱼ in the text dᵢ is predicted.

Note that since Equation (1) is symmetrical with respect to dᵢ and wⱼ, a probability P (dᵢ | wⱼ) that one text dᵢ of m texts is predicted from one word wⱼ of n words may be calculated. Predicting one text dᵢ from one word wⱼ means that, when a certain word wⱼ appears, a possibility of including the word wⱼ in the text dᵢ is predicted.

In Equation (1), an exponential function value is used, where e is the base and the inner product of the word vector w→ and the text vector d→ is the exponent. Then, a ratio of an exponential function value calculated from a combination of a text dᵢ and a word wⱼ to be predicted to the sum of n exponential function values calculated from each combination of the text dᵢ and n words wₖ (k = 1, 2, ..., n) is calculated as a correct answer probability that one word wⱼ is expected from one text dᵢ.

Here, the inner product value of the word vector wⱼ→ and the text vector dᵢ→ can be regarded as a scalar value when the word vector wⱼ→ is projected in a direction of the text vector dᵢ→, that is, a component value in the direction of the text vector dᵢ→ included in the word vector wⱼ→, which can be considered to represent a degree at which the word wⱼ contributes to the text dᵢ. Therefore, obtaining the ratio of the exponential function value calculated for one word Wⱼ to the sum of the exponential function values calculated for n words wₖ (k = 1, 2, ..., n) using the exponential function value calculated using the inner product corresponds to obtaining the correct answer probability that one word wⱼ of n words is predicted from one text dᵢ.

Note that here, a calculation example using the exponential function value using the inner product value of the word vector w→ and the text vector d→ as an exponent has been described. However, the exponential function value may not be used. Any calculation formula using the inner product value of the word vector w→ and the text vector d→ may be used. For example, the probability may be obtained from the ratio of the inner product values itself.

Next, the vector computation unit 42 computes the text vector dᵢ→ and the word vector wⱼ→ that maximize a value L of the sum of the probability P (wⱼ | dᵢ) computed by Equation (1) for all the set S as shown in the following Equation (2). That is, the text vector computation unit 42A and the word vector computation unit 42B compute the probability P (wⱼ | dᵢ) computed by Equation (1) for all combinations of the m texts and the n words, and compute the text vector dᵢ→ and the word vector wⱼ→ that maximize a target variable L using the sum thereof as the target variable L.

### Equation 2

$$\text{L} = {\sum }_{\text{d} ∈ \text{D}} {\sum }_{\text{w} ∈ \text{W}} \# \left(\text{w,d}\right) \text{p} \left(\left(\text{w}\right| \text{d}\right) \qquad \text{(2)}$$

Maximizing the total value L of the probability P (wⱼ | dᵢ) computed for all the combinations of the m texts and the n words corresponds to maximizing the correct answer probability that a certain word wⱼ (j = 1, 2, ..., n) is predicted from a certain text dᵢ (i = 1, 2, ..., m). That is, the vector computation unit 42 can be considered to compute the text vector dᵢ→ and the word vector wⱼ→ that maximize the correct answer probability.

Here, in the present embodiment, as described above, the vector computation unit 42 converts each of the m texts dᵢ into a q-dimensional vector to compute the m texts vectors dᵢ→ including the q axis components, and converts each of the n words into a q-dimensional vector to compute the n word vectors wⱼ→ including the q axis components, which corresponds to computing the text vector dᵢ→ and the word vector wⱼ→ that maximize the target variable L by making q axis directions variable.

The index value computation unit 43 takes each of the inner products of the m text vectors dᵢ→ and the n word vectors wⱼ→ computed by the vector computation unit 42, thereby computing index values reflecting the relationship between the m texts di and the n words wⱼ. In the present embodiment, as shown in the following Equation (3), the index value computation unit 43 obtains the product of a text matrix D having the respective q axis components (d₁₁ to d_{mq}) of the m text vectors dᵢ→ as respective elements and a word matrix W having the respective q axis components (w₁₁ to w_{nq}) of the n word vectors wⱼ→ as respective elements, thereby computing an index value matrix DW having m × n index values as elements. Here, W^{t} is the transposed matrix of the word matrix.

### Equation 3

$$\text{D} = \left(\begin{matrix}{\text{d}}_{11} & {\text{d}}_{12} & ⋯ & {\text{d}}_{1 \text{q}} \\ {\text{d}}_{21} & {\text{d}}_{22} & ⋯ & {\text{d}}_{2 \text{q}} \\ ⋮ & ⋮ & ⋱ & ⋮ \\ {\text{d}}_{\text{m} 1} & {\text{d}}_{\text{m} 2} & ⋯ & {\text{d}}_{\text{mq}}\end{matrix}\right)$$

$$\text{W} = \left(\begin{matrix}{\text{w}}_{11} & {\text{w}}_{12} & ⋯ & {\text{w}}_{1 \text{q}} \\ {\text{w}}_{21} & {\text{w}}_{22} & ⋯ & {\text{w}}_{2 \text{q}} \\ ⋮ & ⋮ & ⋱ & ⋮ \\ {\text{w}}_{\text{n} 1} & {\text{w}}_{\text{m} 2} & ⋯ & {\text{w}}_{\text{mq}}\end{matrix}\right)$$

$$\text{DW} = \text{D} ∗ {\text{W}}^{\text{t}} = \left(\begin{matrix}\text{d} {\text{w}}_{11} & \text{(3)} {\text{w}}_{12} & ⋯ & \text{d} {\text{w}}_{1 \text{n}} \\ \text{d} {\text{w}}_{21} & \text{d} {\text{w}}_{22} & ⋯ & \text{d} {\text{w}}_{2 \text{n}} \\ ⋮ & ⋮ & ⋱ & ⋮ \\ \text{d} {\text{w}}_{\text{m} 1} & \text{d} {\text{w}}_{\text{m} 2} & ⋯ & \text{d} {\text{w}}_{\text{mn}}\end{matrix}\right)$$

Each element of the index value matrix DW computed in this manner may indicate which word contributes to which text and to what extent and which text contributes to which word and to what extent. For example, an element dw₁₂ in the first row and the second column may be a value indicating a degree at which the word w₂ contributes to a text d₁ and may be a value indicating a degree at which the text d₁ contributes to a word w₂. In this way, each row of the index value matrix DW can be used to evaluate the similarity of a text, and each column can be used to evaluate the similarity of a word.

The feature vector specification unit 44 specifies, as a disease feature vector, a word index value group including m index values for one disease name for each of a plurality of disease names among n words. That is, as illustrated in FIG. 6(a), the feature vector specification unit 44 specifies, as a disease feature vector corresponding to each disease name, a word index value group related to a word corresponding to a disease name among n sets of word index value groups (m index values per column) constituting respective columns of the index value matrix DW.

The feature vector specification unit 44 specifies, as a molecule feature vector, a word index value group including m index values for one molecule name for each of a plurality of molecule names among n words. Specifically, as illustrated in FIG. 6(b), the feature vector specification unit 44 specifies, as a molecule feature vector corresponding to each molecule name, a word index value group related to a word corresponding to a molecule name among n sets of word index value groups (m index values per column) constituting respective columns of the index value matrix DW.

Next, a specific description will be given of an example of a pathway generation method.

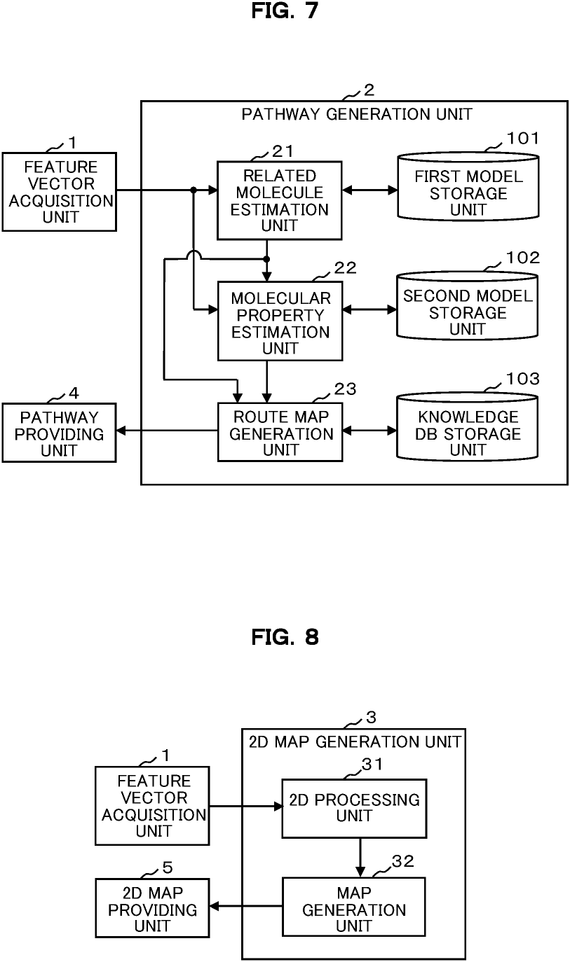

FIG. 7 is a block diagram illustrating a specific functional configuration example of the pathway generation unit 2 according to the first embodiment. As illustrated in FIG. 7, the pathway generation unit 2 according to the first embodiment includes a related molecule estimation unit 21, a molecular property estimation unit 22, and a route map generation unit 23 as functional configurations. Further, the pathway generation unit 2 according to the first embodiment includes a first model storage unit 101, a second model storage unit 102, and a knowledge DB storage unit 103 as storage media.

Each of the above functional blocks 21 to 23 can be configured by any of hardware, DSP, and software. For example, in the case of being configured by software, each of the above functional blocks 21 to 23 actually include a CPU, a RAM, a ROM, etc., of a computer, and is implemented by operating a program stored in a recording medium such as a RAM, a ROM, a hard disk, or a semiconductor memory.

The related molecule estimation unit 21 inputs a disease feature vector acquired by the feature vector acquisition unit 1 for a disease to be analyzed to a first trained model stored in advance in the first model storage unit 101, thereby estimating a plurality of molecules associated with the disease.

A form of the first trained model stored in the first model storage unit 101 may be any of a regression model, a tree model, a neural network model, a Bayesian model, a clustering model, etc. Note that the models listed here are merely examples, and the first trained model is not limited thereto. For example, it is possible to adopt a function model that computes a similarity between a disease feature vector and a molecule feature vector and outputs information about a molecule corresponding to the molecule feature vector whose similarity to the disease feature vector is equal to or more than a predetermined value.

Here, the first trained model is subjected to machine learning so as to output information about a molecule corresponding to the molecule feature vector similar to the disease feature vector when the disease feature vector is input based on a similarity between the disease feature vector and the molecule feature vector. That is, the feature vector computation apparatus illustrated in FIG. 6 computes a disease feature vector related to a plurality of disease names and computes a molecule feature vector related to a plurality of molecule names. Then, machine learning is performed in advance using these data sets, and the first trained model learned based on the similarity between the disease feature vector and the molecule feature vector is stored in the first model storage unit 101.

The fact that the disease feature vector and the molecule feature vector are similar to each other means that a property indicating a text to which a word as a disease name contributes and a degree at which the word contributes to the text is similar to a property indicating a text to which a word as a molecule name contributes and a degree at which the word contributes to the text. Since the text is described according to a specific theme, the disease name and the molecule name, which have a similar relationship between the disease feature vector and the molecule feature vector, mean that contributions to a plurality of texts described in relation to each theme are similar, and it is possible to presume that there is some association between the disease and the molecule.

In case that the disease name and the molecule name are described in one text, it is clear that the disease and the molecule are related. On the other hand, when the disease name and the molecule name are described across a plurality of texts, it is unclear whether there is relevance between a disease described in one text and a molecule described in another text, and even when medical personnel read these texts, it is difficult to immediately understand that there is relevance.

On the other hand, according to the present embodiment, even when a disease name and a molecule name are described across a plurality of texts in this way, it is possible to presume that there may be some relevance between the disease and the molecule. In this way, when a disease feature vector corresponding to a certain disease name is input to the first trained model, even a molecule whose relevance to the disease is unknown may be output as a related molecule by estimation based on learning.

Note that instead of the configuration including the related molecule estimation unit 21 and the first model storage unit 101, the pathway generation unit 2 may have a configuration for specifying a molecule (molecule name) corresponding to a plurality of molecule feature vectors acquired by the feature vector acquisition unit 1 from the plurality of molecule feature vectors with reference to a database (not illustrated) associating and storing the molecule name with the corresponding molecule feature vector. Alternatively, the feature vector acquisition unit 1 may include the related molecule estimation unit 21 and the first model storage unit 101 as a configuration for the feature vector acquisition unit 1 to acquire a plurality of molecule feature vectors.

The molecular property estimation unit 22 inputs a disease feature vector acquired by the feature vector acquisition unit 1 for a disease to be analyzed and a molecule feature vector specified for a plurality of molecules estimated by the related molecule estimation unit 21 to the second trained model stored in the second model storage unit 102, thereby estimating a probability that a molecule acting on the disease is causative or responsive as a property for each of a plurality of molecules presumed to be associated with the disease.

A form of the second trained model stored in the second model storage unit 102 may be any of a regression model, a tree model, a neural network model, a Bayesian model, a clustering model, etc. Note that the models listed here are merely examples, and the second trained model is not limited thereto.

Here, the second trained model is subjected to machine learning so as to output a probability that a property of a molecule is causative or responsive when a disease feature vector and a molecule feature vector are input using the disease feature vector, the molecule feature vector, and a data set of property information representing the property of the molecule acting on a disease as teacher data. The causativeness is a property that may cause a disease due to the presence or mutation of the molecule. Responsiveness is a property that a molecule may mutate due to the onset of a disease. In the present embodiment, as an example, the second trained model will be described as outputting a probability that a property of a molecule with respect to a disease is causative.

With regard to a known disease, there is known information about which molecule is causative and which molecule is responsive. The second trained model is created by setting a disease feature vector, a molecule feature vector, and a data set of property information of a molecule generated from this known information as teacher data (property information of a molecule is set as correct answer data) and performing machine learning using this data set. Therefore, for a molecule whose property is known to be causative for a known disease, a high probability value is output from the second trained model. On the other hand, for a molecule whose property is known to be responsive for a known disease, a low probability value is output from the second trained model.

In addition, a molecule whose relevance to a disease is unknown in human knowledge so far may be included in a plurality of molecules whose relevance to the disease is estimated by the related molecule estimation unit 21. Even for such a molecule, by estimation based on learning, a value of a probability indicating that that molecule may exhibit a causative property with respect to the disease is output from the second trained model.

That is, in case that a similarly between (a feature quantity obtained from) a combination of a disease feature vector corresponding to a certain disease and a molecule feature vector corresponding to a molecule whose relevance to the disease is unknown and (a feature quantity obtained from) a combination of a disease feature vector corresponding to the certain disease and a molecule feature vector corresponding to a molecule known to be causative is high, a relatively high probability value tends to be output from the second trained model.

On the other hand, in case that a similarly between (a feature quantity obtained from) a combination of a disease feature vector corresponding to a certain disease and a molecule feature vector corresponding to a molecule whose relevance to the disease is unknown and (a feature quantity obtained from) a combination of a disease feature vector corresponding to the certain disease and a molecule feature vector corresponding to a molecule known to be responsive is high, a relatively low probability value tends to be output from the second trained model.

As described above, the molecular property estimation unit 22 estimates the property of the action of the molecule on the disease by inputting the disease feature vector and the molecule feature vector to the second trained model. Here, as the disease feature vector, one acquired by the feature vector acquisition unit 1 is used. On the other hand, as the molecule feature vector, a plurality of molecules estimated by the related molecule estimation unit 21, that is, a molecule feature vector corresponding to a molecule information list output from the first trained model is used.

With regard to the molecule feature vector, for example, the molecular property estimation unit 22 reads a molecule feature vector corresponding to a molecule name estimated by the related molecule estimation unit 21 from a database (not illustrated) that associates and stores a molecule name with a molecule feature vector corresponding thereto. Note that the molecular property estimation unit 22 may compute the molecule feature vector from molecular names thereof in real time when a molecule information list is output from the first trained model. That is, the molecular property estimation unit 22 may have a function of the feature vector computation apparatus described above and specify a molecule feature vector by executing the function of the feature vector computation apparatus.

As another example, as the first trained model used in the related molecule estimation unit 21, it is possible to use one subjected to machine learning so as to output a molecule feature vector similar to a disease feature vector when the disease feature vector is input based on a similarity between the disease feature vector and the molecule feature vector. In this case, the molecular property estimation unit 22 can input the disease feature vector output from the feature vector acquisition unit 1 and the molecule feature vector output from the related molecule estimation unit 21 to the second trained model directly.

The route map generation unit 23 generates a pathway representing an intermolecular interaction as a route map in a manner that a causative molecule is on an upstream side and a responsive molecule is on an downstream side and that an intermolecular connection relationship shown by a known knowledge database is reflected for a plurality of molecules whose relevance to the disease is estimated by the related molecule estimation unit 21 by using a property of a molecule estimated by the molecular property estimation unit 22 and the knowledge database showing the intermolecular connection relationship.

In this instance, for example, the route map generation unit 23 generates the pathway in a manner that a molecule (hereinafter referred to as a causative molecule) whose probability value estimated to be causative by the molecular property estimation unit 22 is larger than a first threshold Th1 is disposed on the upstream side of the pathway, a molecule (hereinafter referred to as a responsive molecule) whose probability value is smaller than a second threshold Th2 (Th1 > Th2) is disposed on the downstream side of the pathway, and a molecule (hereinafter referred to as a linking molecule) whose probability value is larger than or equal to the second threshold Th2 and smaller than or equal to the first threshold Th1 is disposed between the causative molecule and the responsive molecule.

The known knowledge database showing the intermolecular connection relationship is stored in advance in the knowledge DB storage unit 103. For example, the intermolecular connection relationship includes a relationship in which when an expression level of a certain molecule increases (or decreases), an expression level of another molecule increases (decreases) in conjunction with the increase (decrease). The knowledge DB storage unit 103 stores in advance known information about such an intermolecular relationship. However, the known information contained in the knowledge database stored in the knowledge DB storage unit 103 is limited to information indicating which molecule has a relationship with which molecule, and does not include information indicating the magnitude of the connection relationship. In addition, the known information is a set of information showing a connection relationship between two molecules, and is not information showing a sequential connection relationship between three or more molecules.

On the other hand, for example, the route map generation unit 23 supplements the magnitude of the intermolecular relationship not contained in the known information by using a value of a degree of similarity between the disease feature vector and the molecule feature vector specified by the related molecule estimation unit 21. For example, it is possible to generate a pathway by facilitating the connection between these molecules on the assumption that the molecules having similar similarity values have a strong relationship with each other. In addition, for example, the route map generation unit 23 uses a minimum flow algorithm to specify a sequential connection relationship between three or more molecules.

As described above, the route map generation unit 23 generates a pathway that represents an interaction between three or more molecules as a route map by setting the causative molecule on the upstream side and the responsive molecule on the downstream side, and reflecting the connection relationship shown by the knowledge database. Note that even though an example of using the minimum flow algorithm has been described here, the invention is not limited thereto.

In the pathway illustrated in FIG. 3, a diamond-shaped symbol mainly shown on the upstream side of the pathway is the causative molecule, a square symbol mainly shown on the downstream side is the responsive molecule, and an elliptical symbol is the linking molecule. Even though a molecule name is not written on each symbol for convenience of drawing, the molecule name is actually displayed on each symbol.

As mentioned above, this pathway may include a molecule whose relevance to a disease is unknown, and may include connectivity (intermolecular interaction) in which a property of a molecule with respect to the disease is unknown. By viewing such a pathway, for a disease to be analyzed, the user of the client terminal 20 can easily detect a certain molecule that may be related to the disease or a certain molecule that may be affected when a molecule is operated and the molecule that affects the certain molecule.

That is, according to the present embodiment, when a disease feature vector for a disease to be analyzed is input to the first trained model, not only a molecule known to be related to the disease, but also a molecule whose relevance to the disease is unknown may be output as a related molecule by estimation based on learning. In addition, when a molecule feature vector of the molecule estimated in this way and the disease feature vector are input to the second trained model, a probability indicating whether a molecule may exhibit a causative or responsive property with respect to the disease is output not only for the molecule whose relevance to the disease is known but also the molecule whose relevance is unknown by estimation based on learning. Then, an estimation result with regard to a property of a molecule presumed to be associated with the disease in this way and the known knowledge database showing the intermolecular connection relationship are used to generate a pathway representing an intermolecular interaction as a route map.

As described above, according to the present embodiment, it is possible to generate a pathway useful for obtaining new knowledge beyond a range of a known intermolecular interaction described in a literature, etc., and the pathway can be effectively used for research and development of treatment, drug discovery, etc. of a disease.

Note that here, a description has been given of an example in which the first trained model and the second trained model are created in advance and stored in the first model storage unit 101 and the second model storage unit 102. The apparatus that performs machine learning may be configured as an apparatus different from the server apparatus 10, or the server apparatus 10 may be configured to have a function of performing machine learning.

Further, a description has been given of an example of generating a pathway using a plurality of molecule feature vectors similar to a disease feature vector of a disease name designated by the user. However, the pathway may be generated using molecule feature vectors at a plurality of positions plotted on a 2D map. For example, when a pathway acquisition request is transmitted to the server apparatus 100 in a state in which a 2D map is generated and displayed on the client terminal 20, the pathway generation unit 2 may automatically designate the entire range of the 2D map, specify molecules corresponding to molecule feature vectors at a plurality of positions existing in the designated range, and generate a pathway using the specified molecules.

Next, a specific description will be given of an example of a generation method for a 2D map.

FIG. 8 is a block diagram illustrating a specific functional configuration example of the 2D map generation unit 3 according to the first embodiment. As illustrated in FIG. 8, the 2D map generation unit 3 according to the first embodiment includes a 2D processing unit 31 and a map generation unit 32 as functional configurations.

The 2D processing unit 31 generates 2D coordinate information by performing a dimension compression process on a plurality of molecule feature vectors acquired by the feature vector acquisition unit 1. That is, the 2D processing unit 31 replaces multidimensional component information included in each of the plurality of molecule feature vectors with 2D coordinate information including an x-coordinate and a y-coordinate, respectively.

For example, when it is presumed that the number of a plurality of molecule feature vectors acquired by the feature vector acquisition unit 1 is k and the number of dimensions of the molecule feature vector (the number of components of the molecule feature vector) is q, respective component values of the k molecule feature vectors can be represented as a matrix of k rows × q columns (hereinafter referred to as a feature vector matrix). The 2D processing unit 31 performs a known dimension compression process on this feature vector matrix to dimensionally compress the matrix into a matrix of k rows × 2 columns. Values in these two columns are information about 2D coordinates (x, y). As the known dimension compression process, for example, it is possible to use principal component analysis (PCA), singular value decomposition (SVD), etc.

In this way, by compressing the dimension of the feature vector matrix using the PCA or SVD method, the feature vector matrix can be low-rank approximated without damaging the features of the respective molecule feature vectors represented by the feature vector matrix as much as possible.

The map generation unit 12 generates a 2D map in which a plurality of molecule feature vectors is plotted in an x-y 2D coordinate system based on a plurality of pieces of 2D coordinate information generated by the 2D processing unit 31 for a plurality of molecule feature vectors. In the 2D map illustrated in FIG. 4, each position specified by a plurality of pieces of 2D coordinate information generated from a plurality of molecule feature vectors is plotted on a 2D plane. As described above, in the first embodiment, the number of positions plotted on the 2D map illustrated in FIG. 4 is the same as the number of all symbols appearing on the pathway illustrated in FIG. 3.

As described above, in the first embodiment, when the user designates an arbitrary disease name on the client terminal 20 and sends a pathway acquisition request or a 2D map acquisition request to the server apparatus 10, it is possible to acquire pathway data or 2D map data for a plurality of molecules related to the designated disease from the server apparatus 10, and display the pathway of FIG. 3 or the 2D map of FIG. 4 on the client terminal 20. It is possible to display both the pathway and the 2D map. Further, by transmitting a 2D map acquisition request to the server apparatus 10 in a state in which a pathway has been generated, or by transmitting a pathway acquisition request to the server apparatus 10 in a state in which a 2D map has been generated, it is possible to display both the pathway and the 2D map.

The pathway generated in the present embodiment is not simply obtained by visualizing known content described in a literature by a human or a computer as a route map, and is generated and visualized by utilizing a property of information of each of a plurality of molecule feature vectors. Further, in the 2D map generated in the present embodiment, a coordinate position is determined according to a property of information of each of a plurality of feature vectors, so that feature vectors having similar properties are disposed at positions close to each other, and feature vectors having dissimilar properties are disposed at positions far apart from each other. By using a pathway and a 2D map having such properties together, the user can perform information analysis useful for obtaining new knowledge beyond a range of known knowledge described in a literature, etc.

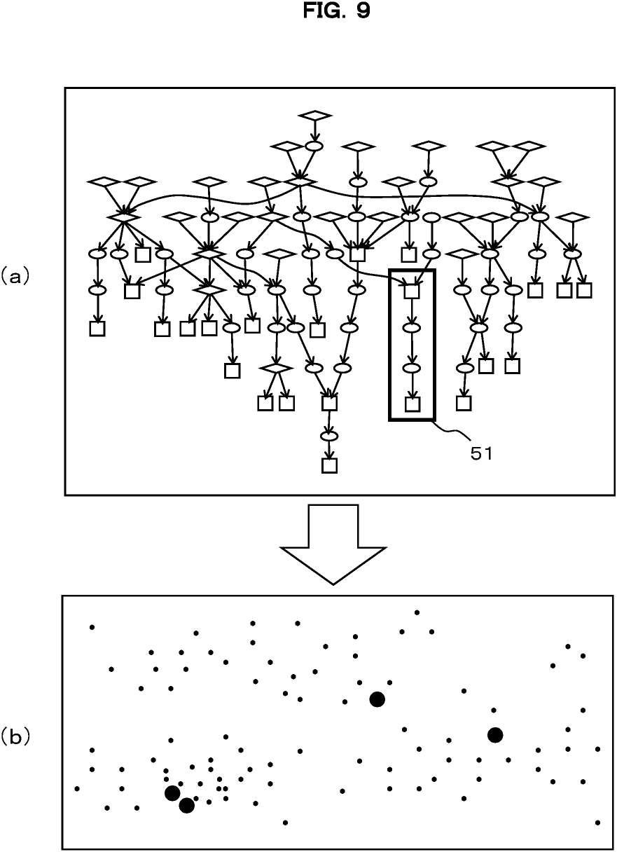

Here, when an arbitrary route is designated in the pathway generated by the pathway generation unit 2, the 2D map generation unit 3 may display a position of a molecule included in the path on a 2D map in manner that the position can be distinguished from a position of another molecule. FIG. 9 is a diagram illustrating an example of a linked display between the pathway and the 2D map in this case. FIG. 9(a) illustrates a state in which the user operates a mouse, etc. to designate an arbitrary route by a rectangular frame 51 in a pathway displayed on the client terminal 20. A plurality of molecules is included in the route designated by the rectangular frame 51. Note that a method of designating the route is not limited to a method using the rectangular frame 51.

FIG. 9(b) illustrates a state in which positions of a plurality of molecules included in a route designated by the rectangular frame 51 are emphasized (for example, changed in size) and displayed in a 2D map displayed on the client terminal 20. In an example illustrated in FIG. 9(b), molecules present at positions close to each other and molecules present at positions far apart from each other are emphasized and displayed on the 2D map. In the way, the user can detect that molecules having similar properties of feature vectors and molecules having dissimilar properties of feature vectors are present in a plurality of molecules shown to have a connection relationship on one route in a pathway. Furthermore, in the 2D map illustrated in FIG. 9(b), the user can detect other molecules around an emphasized and displayed molecule, that is, other molecules having similar properties of feature vectors.

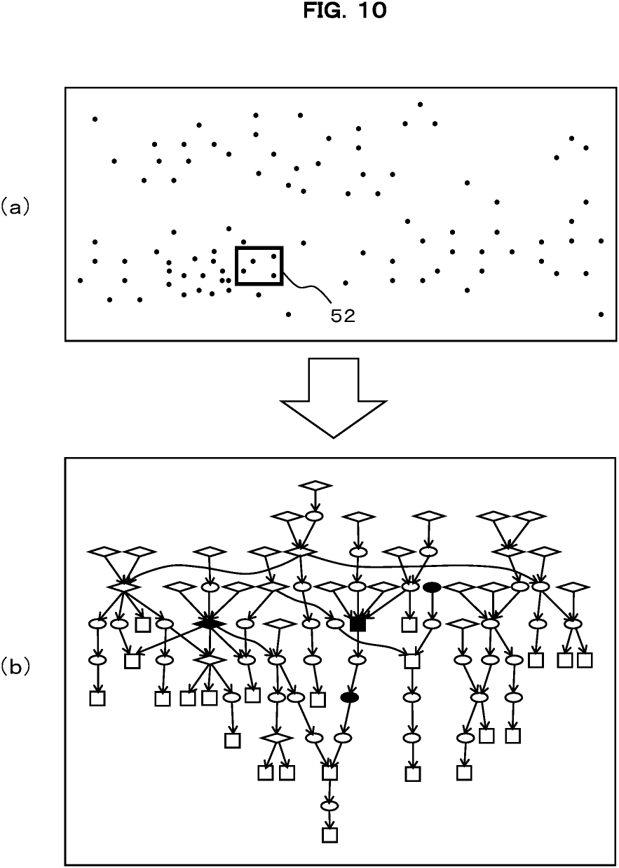

Further, when an arbitrary position or range is designated in a 2D map generated by the 2D map generation unit 3, the pathway generation unit 2 may display a position of one or more molecules on a pathway corresponding to the designated position or range in manner that the position is distinguishable from a position of another molecule. FIG. 10 is a diagram illustrating an example of a linked display of the pathway and the 2D map in this case. FIG. 10(a) illustrates a state in which the user operates the mouse, etc. to designate an arbitrary range by a rectangular frame 52 in a 2D map displayed on the client terminal 20. A path designated by the rectangular frame 52 includes plots corresponding to a plurality of molecules.

FIG. 10(b) illustrates a state in which positions of a plurality of molecules included in a range designated by the rectangular frame 52 are emphasized (for example, changed in color) and displayed in a pathway displayed on the client terminal 20. In an example illustrated in FIG. 10(b), molecules existing on a plurality of different routes are emphasized and displayed. As a result, the user can detect that molecules existing on the same route and having a connection relationship and molecules existing on different routes and not having a connection relationship are present among a plurality of molecules disposed close to each other in a 2D map and shown to be similar in property to each other.

Here, as described above, a molecule feature vector is information including a plurality of word index value groups representing a text to which a word as a molecule name contributes and a degree at which the word contributes to the text. Therefore, 2D coordinate information generated from a molecule feature vector and text-related information of a text shown to be contributed by a word index value group included in the molecule feature vector may be associated with each other and stored in a database, and text-related information corresponding to a position designated by the user on a 2D map may be displayed on the client terminal 20. Similarly, position information of each symbol on a pathway and text-related information of a text shown to be contributed by a word index value group included in a molecule feature vector corresponding to each symbol may be associated with each other and stored in a database, and text-related information corresponding to a symbol designated by the user on the pathway may be displayed on the client terminal 20.

Here, the position on the 2D map and the symbol on the pathway can be individually designated by the user operating a mouse, a touch panel, etc. In this case, the text-related information corresponding to the individually designated position is displayed on the client terminal 20. Further, the user may operate the mouse, the touch panel, etc. to designate a range on the 2D map or the pathway by the rectangular frames 51 and 52. In this case, one or more pieces of text-related information corresponding to molecules included in the designated rectangular frames 51 and 52 are displayed in a list on the client terminal 20. The text-related information may be the actual data of a text, or may be information that can specify a text, such as a source or a title of the text.

In this way, for example, when the user designates a molecule emphasized and displayed on the 2D map or a molecule around the molecule, or when a molecule emphasized and displayed on the pathway or a molecule on the same route as that of the molecule is designated, it is possible to detect a text related to the designated molecule, and it is possible to obtain information useful for research and development of treatment, drug discovery, etc. of a disease by confirming the text. For example, it is possible to discover concomitant drug candidates for efficiently blocking a plurality of paths and to predict efficacy and safety of drug administration while being able to discover a possibility of treatment using an existing drug and a new target or biomarker which is inconceivable in existing knowledge.

### Second Embodiment

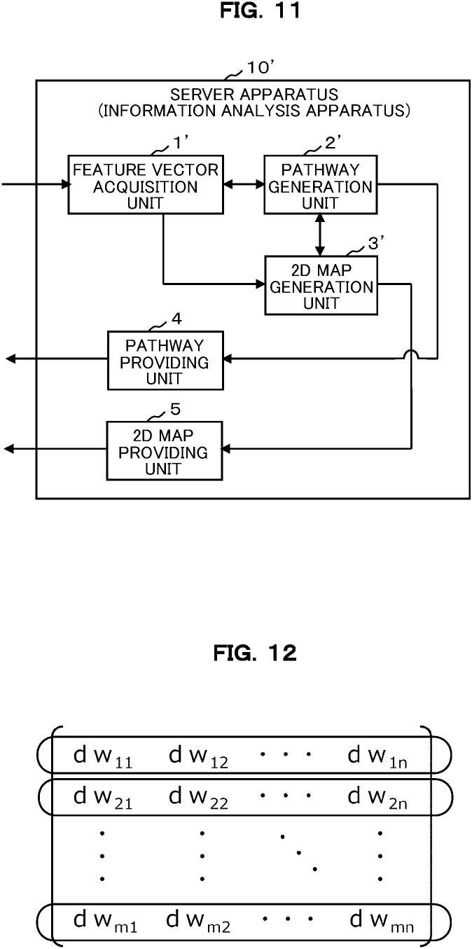

Next, a second embodiment of the invention will be described with reference to the drawings. An overall configuration of an information providing system including an information analysis apparatus according to the second embodiment is similar to that of FIG. 1. However, a server apparatus 10′ is used instead of the server apparatus 10. FIG. 11 is a block diagram illustrating a functional configuration example of the server apparatus 10′ (information analysis apparatus) according to the second embodiment. In FIG. 11, those having the same reference symbols as those illustrated in FIG. 2 have the same functions, and therefore, duplicate description will be omitted here.

As illustrated in FIG. 11, the server apparatus 10′ according to the second embodiment includes a feature vector acquisition unit 1′, a pathway generation unit 2′, and a 2D map generation unit 3′ as functional configurations instead of the feature vector acquisition unit 1, the pathway generation unit 2, and the 2D map generation unit 3 illustrated in FIG. 2.

The feature vector acquisition unit 1′ acquires the disease feature vector and the molecule feature vector described in the first embodiment. In the first embodiment described above, only the disease feature vector corresponding to the disease name designated by the user was acquired. However, in the second embodiment, all the plurality of disease feature vectors computed by the feature vector computation apparatus of FIG. 5 by using a plurality of texts to be analyzed is acquired. Further, in the first embodiment described above, only the molecule feature vector similar to the disease feature vector corresponding to the disease name designated by the user is acquired. However, in the second embodiment, all the plurality of molecule feature vectors computed from the plurality of texts to be analyzed is acquired.

As a plurality of word feature vectors specified for each of a plurality of words included in a plurality of texts, the feature vector acquisition unit 1′ may acquire a plurality of feature vectors related to at least one type of name among a plurality of word feature vectors specified for a name of a drug (hereinafter referred to as a drug feature vector), a plurality of word feature vectors specified for a name of a compound (hereinafter referred to as a compound feature vector), and a plurality of word feature vectors specified for a name of a metabolite (hereinafter referred to as a metabolite feature vector) in addition to the disease feature vector and the molecule feature vector.

The feature vector acquisition unit 1′ may further acquire a plurality of text feature vectors specified for each of a plurality of texts. As illustrated in FIG. 12, a text feature vector corresponds to a text index value group (n index values per row) including m sets of index values included in each row of an index value matrix DW computed by an index value computation unit 43 of FIG. 5, and is specified by a feature vector specification unit 44.

A word feature vector such as a disease feature vector, a molecule feature vector, a drug feature vector, a compound feature vector, or a metabolite feature vector is a vector representing a text to which a word included in a plurality of texts contributes and a degree at which the word contributes to the text, and a text feature vector is a vector representing a word among a plurality of words included in a plurality of texts to which a text contributes and a degree at which the text contributes to the word. Each feature vector is specified as an index value group in a row direction and an index value group in a column direction in one index value matrix DW computed from a plurality of texts to be analyzed, and has a relationship that the vectors are generated from the same analysis target.

In the second embodiment, first, the 2D map generation unit 3′ generates a 2D map. Thereafter, the pathway generation unit 2′ specifies a plurality of molecules as a word using a position or a range designated in the 2D map generated by the 2D map generation unit 3′, and generates a pathway representing an intermolecular interaction as a route map using a knowledge database showing an intermolecular connection relationship for the specified plurality of molecules.

The 2D map generation unit 3′ generates at least one type of 2D map using a plurality of feature vectors (word feature vectors or text feature vectors) related to at least one type of name or text acquired by the feature vector acquisition unit 1′. A generation method for the 2D map using the text feature vectors is similar to that of the first embodiment. That is, the 2D map generation unit 3′ generates a plurality of pieces of 2D coordinate information by performing dimension compression on a plurality of text feature vectors acquired by the feature vector acquisition unit 1′, and generates a 2D map in which positions corresponding to the plurality of text feature vectors are visualized on a 2D plane based on the generated plurality of pieces of 2D coordinate information.

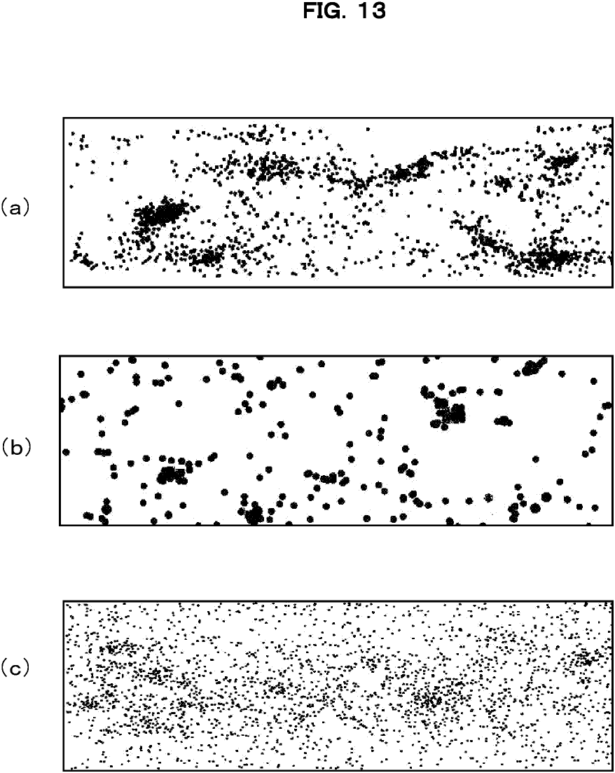

FIG. 13 is a diagram illustrating an example of a plurality of types of 2D maps generated by the 2D map generation unit 3′. FIG. 13(a) illustrates a 2D map (hereinafter referred to as a molecule 2D map) generated based on a plurality of molecule feature vectors. FIG. 13(b) illustrates a 2D map (hereinafter referred to as a disease 2D map) generated based on a plurality of disease feature vectors. FIG. 13(c) illustrates a 2D map (hereinafter referred to as a text 2D map) generated based on a plurality of text feature vectors.

The pathway generation unit 2′ specifies a plurality of molecules including one or more molecules corresponding to a designated position or range in a molecule 2D map generated by the 2D map generation unit 3′ and one or more other molecules whose connection relationship with one or more molecules is shown by a knowledge database, and generates a pathway for the specified plurality of molecules.

For example, an arbitrary position or range on the 2D map may be designated by the user operating the mouse, the touch panel, etc. on the 2D map displayed on the client terminal 20. For example, arbitrary one or more positions among a plurality of positions plotted on the 2D map can be designated by a user operation, and the pathway generation unit 2′ specifies one or more molecules corresponding to the designated positions. Here, by inputting arbitrary one or more molecule names as a search keyword on the client terminal 20 on which the 2D map is displayed, a plot position of a molecule feature vector corresponding to the input molecule name may be emphasized and displayed, and the emphasized and displayed position may be designated by a user operation. Further, on the 2D map, an arbitrary range may be designated by a user operation of setting a frame having an arbitrary shape, and the pathway generation unit 2′ specifies one or more molecules included in this designated range.

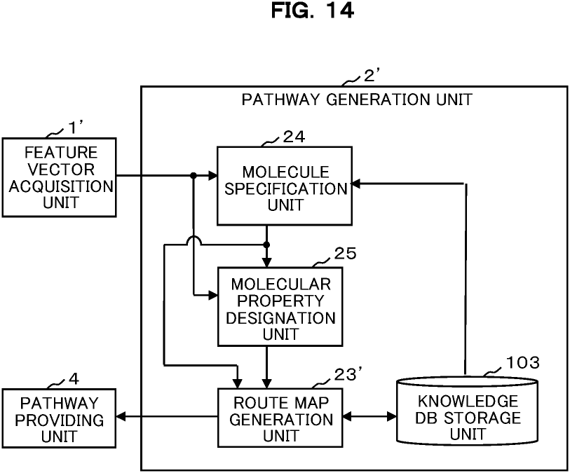

FIG. 14 is a block diagram illustrating a specific functional configuration example of the pathway generation unit 2′ according to the second embodiment. In FIG. 14, those having the same reference symbols as those illustrated in FIG. 7 have the same functions, and therefore, duplicate description will be omitted here. As illustrated in FIG. 14, the pathway generation unit 2′ according to the second embodiment includes a molecule specification unit 24 instead of the related molecule estimation unit 21 and the first model storage unit 101 illustrated in FIG. 7, and includes a molecular property designation unit 25 instead of the molecular property estimation unit 22 and the second model storage unit 102. Further, a route map generation unit 23′ is included instead of the route map generation unit 23.

The molecule specification unit 24 specifies one or more molecules corresponding to a position or a range designated on a 2D map by a user operation on the client terminal 20 among a plurality of molecules corresponding to a plurality of molecule feature vectors acquired by the feature vector acquisition unit 1′. Further, the molecule specification unit 24 further specifies one or more other molecules whose connection relationship with the one or more specified molecules described above using a knowledge database showing an intermolecular connection relationship stored in the knowledge DB storage unit 103.

The molecular property designation unit 25 designates some molecules as causative molecules and some molecules as responsive molecules for some or all of a plurality of molecules specified by the molecule specification unit 24 according to a user operation on the client terminal 20. For example, a list of a plurality of molecules specified by the molecule specification unit 24 is displayed on the display apparatus of the client terminal 20, and the user is allowed to designate some molecules as causative molecules and some molecules as responsive molecules. This designation is performed using a mouse, a touch panel, etc. The user designates causativeness or responsiveness of a molecule whose causativeness or responsiveness is known for a disease to be analyzed.

Note that a method of designating causativeness or responsiveness is not limited thereto. For example, the following configuration can be adopted. That is, the molecular property designation unit 25 stores in advance known information about which molecule is causative and which molecule is responsive with respect to a known disease in a database. Then, in the client terminal 20, the user designates a disease to be analyzed. In response to this designation, the molecular property designation unit 25 designates causativeness or responsiveness of a molecule stored for the designated disease with reference to the database described above. Alternatively, the molecular property estimation unit 22 and the second model storage unit 102 may be included instead of the molecular property designation unit 25, and a property of a molecule may be estimated similarly to the first embodiment.

Using a knowledge database showing a property of a molecule designated by the molecular property designation unit 25 and an intermolecular connection relationship stored in the knowledge DB storage unit 103, the route map generation unit 23′ generates a pathway representing an intermolecular interaction as a route map by setting a causative molecule on an upstream side and a responsive molecule on a downstream side for a plurality of molecules specified by the molecule specification unit 24, and reflecting a connection relationship shown by a knowledge database.

In the second embodiment, when an arbitrary position or range is designated on a 2Dmap of one type, the 2D map generation unit 3′ may visualize a position or a range on a 2D map of another type corresponding to the designated position or range, or display one or more positions included in a range on a 2D map of another type corresponding to the designated range in manner that the position is distinguishable from another position.

For example, the arbitrary position or range on the 2D map may be designated by the user operating the mouse, the touch panel, etc. on the 2D map displayed on the client terminal 20. Alternatively, a plot position of a feature vector corresponding to an input name can be designated by inputting an arbitrary molecule name, disease name, drug name, compound name or metabolite name as a search keyword.

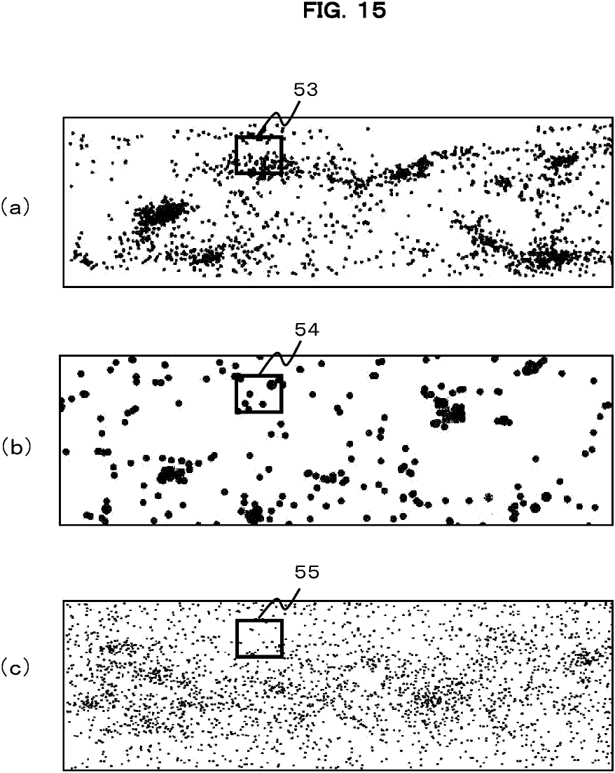

FIG. 15 is a diagram illustrating an example of a linked display of a plurality of types of 2D maps in the second embodiment. FIG. 15(a) illustrates a state in which the user operates the mouse, etc. to designate an arbitrary range by a rectangular frame 53 in a molecule 2D map displayed on the client terminal 20. A plurality of molecules is included in a route designated by the rectangular frame 53. FIGS. 15(b) and 15(c) illustrate states in which ranges corresponding to the range designated by the rectangular frame 53 are visualized by rectangular frames 54 and 55 in a disease 2D map and a text 2D map displayed on the client terminal 20. Instead of visualizing the ranges by the rectangular frames 54 and 55, a position of one or more molecules existing in a range corresponding to the rectangular frames 54 and 55 may be displayed in manner that the position is distinguishable from another position.

As described above, all of a molecule feature vector, a disease feature vector, and a text feature vector which are bases for generating each 2D map illustrated in FIG. 15 are specified as an index value group in a row direction and an index value group in a column direction in one index value matrix DW computed from a plurality of texts to be analyzed. For this reason, for feature vectors at the same position or in the same range or feature vectors at positions close to each other on 2D maps of a plurality of types, it can be inferred that there is a possibility of some relationship therebetween.

By utilizing this fact, it is possible to check molecules, diseases, and texts existing at the same position, in the same range, or at positions close to each other on 2D maps of different types, and consider a relationship between molecules and diseases, etc. using the corresponding texts. Here, the following configuration can be adopted. That is, 2D coordinate information on a molecule 2D map generated from a molecule feature vector and text-related information of a text shown to be contributed by a word index value group included in the molecule feature vector are associated and stored in a database. In addition, 2D coordinate information of a disease 2D map generated from a disease feature vector and text-related information of a text shown to be contributed by a word index value group included in the disease feature vector are associated and stored in a database. Then, text-related information corresponding to a molecule or a disease designated by the user on a 2D map is displayed on the client terminal 20.

FIG. 15 illustrates three types of 2D maps which are the molecule 2D map, the disease 2D map, and the text 2D map. However, similar processing can be applied to a drug 2D map, a compound 2D map, and a metabolite 2D map. In this way, it is possible to consider a relationship among diseases, molecules, drugs, compounds, and metabolites, etc. using the corresponding texts. Furthermore, by using a plurality of types of 2D maps and pathways together, it is possible to consider an intermolecular connection relationship as well. In this way, for example, in addition to the therapeutic potential of existing drugs and the discovery of new targets or biomarkers unthinkable with existing knowledge, with regard to the discovery of concomitant drug candidates for efficiently inhibiting a plurality of paths, and the prediction of the efficacy and safety of drug administration, etc., it is possible to perform information analysis useful for obtaining new knowledge beyond a range of known knowledge.

Note that similarly to the first embodiment, when an arbitrary route is designated in a pathway generated by the pathway generation unit 2′, the 2D map generation unit 3′ may display a position of a molecule included in the route on a molecule 2D map in manner that the position is distinguishable from a position of another molecule. In the second embodiment, not only a molecule forming a pathway, but also other molecules are plotted on a molecule 2D map. For this reason, a molecule not existing on a pathway may be present near a molecule emphasized and displayed on a molecule 2D map. This molecule does not appear on the pathway. However, this molecule is a molecule presumed to have some relationship. It may be possible to obtain new findings by considering this molecule using related texts.

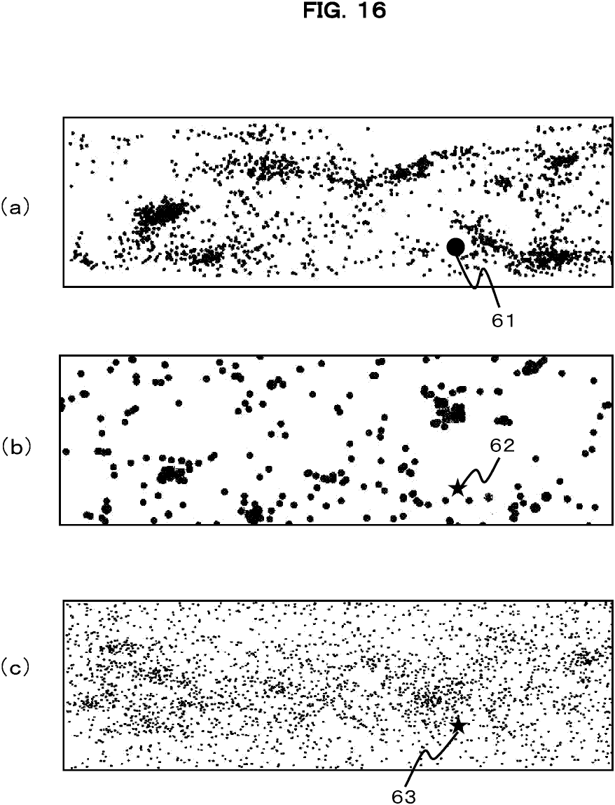

Further, when an arbitrary molecule name, disease name, drug name, compound name, or metabolite name is input as a search keyword (when an arbitrary name related to a 2D map of one type is designated), a plot position of a feature vector corresponding to the search keyword may be displayed (for example, emphasized and displayed) on a 2D map of a type corresponding to the search keyword in manner that the plot position is distinguishable from another position, and a position having the same 2D coordinates as those of the plot position may be visualized (for example, a predetermined mark may be displayed) on a 2D map of another type. FIG. 16 is a diagram illustrating a linked display example of a plurality of types of 2D maps in this case.

FIG. 16(a) illustrates a state in which when the user inputs an arbitrary molecule name as a search keyword, a plot position 61 of a molecule feature vector corresponding to the search keyword is emphasized and displayed in a molecule 2D map displayed on the client terminal 20. FIGS. 16(b) and 16(c) illustrate states in which predetermined marks 62 and 63 are displayed at positions of the same 2D coordinates as those of the plot position 61 emphasized and displayed on the molecule 2D map in the disease 2D map and the text 2D map displayed on the client terminal 20. A plot corresponding to a disease or a text may not be present at positions where the marks 62 and 63 are displayed. However, a plot may be present around the plot. The user can consider a disease or a text at a plot position displayed around the marks 62 and 63.

In the second embodiment described above, a description has been given of an example of specifying one or more molecules corresponding to a position or a range designated on a molecule 2D map. However, the invention is not limited thereto. For example, it is possible to specify one or more molecules corresponding to a designated position or range on a 2D map of a different type from that of the molecule 2D map. For example, the pathway generation unit 2′ may use a disease 2D map and a molecule 2D map to specify one or more molecules at a position or in a range on the molecule 2D map corresponding to a position or a range designated in the disease 2D map, specify a plurality of molecules including one or more other molecules whose connection relationship with the one or more molecules is shown by a knowledge database, and generate a pathway for the specified plurality of molecules.

Further, in the first and second embodiments, a description has been given of an example in which the feature vector computed by the feature vector computation apparatus illustrated in FIG. 5 is used as the word feature vector and the text feature vector. However, the invention is not limited thereto. For example, in case that a vector represents a text to which a word contained in a plurality of texts contributes and a degree at which the disease name or the molecule name contributes to the text, the vector is not limited to the word feature vector computed by the feature vector computation apparatus illustrated in FIG. 5. Further, as long as a vector represents a word among a plurality of words included in a plurality of texts to which a text contributes and a degree at which the text contributes to the word, the vector is not limited to the text feature vector computed by the feature vector computation apparatus illustrated in FIG. 5.

When the feature vector computed by the feature vector computation apparatus illustrated in FIG. 5 is used as the word feature vector and the text feature vector, there are advantages that a plurality of types of word feature vector and a plurality of types of text feature vector can be extracted from one index value matrix DW computed by one algorithm, and mutual similarity or relationship can be more logically specified. Thus, it is possible to improve certainty of an estimation result performed by using the first trained model and the second trained model, and to enhance usefulness of a generated pathway. In addition, for a relationship between plot positions on a plurality of types of 2D maps and a relationship between a 2D map and a pathway, there is an advantage that logical meanings can be made based on feature vectors similar or related to each other.

Further, in the first and second embodiments, it has been described that for a text to be targeted when a disease feature vector and a molecule feature vector are created, without being limited to a description of a disease, a description of various other themes may be included. However, the invention is not limited thereto. For example, only a text containing description content related to a specific disease may be targeted.

In addition, the first and second embodiments are merely examples of embodiment in carrying out the invention, and the technical scope of the invention should not be construed in a limited manner by the embodiments. That is, the invention can be implemented in various forms without departing from a gist or a main feature thereof.

### REFERENCE SIGNS LIST

10, 10′ Server apparatus (information analysis apparatus)
1 Feature vector acquisition unit
2, 2′ Pathway generation unit
3, 3′ 2D map generation unit
21 Related molecule estimation unit
22 Molecular property estimation unit
23, 23′ Route map generation unit
24 Molecule specification unit
25 Molecular property designation unit
31 2D processing unit
32 Map generation unit
101 First model storage unit
102 Second model storage unit
103 Knowledge DB storage unit

1. An information analysis apparatus characterized by comprising:
a feature vector acquisition unit that acquires a plurality of feature vectors including a plurality of word feature vectors specified for each of a plurality of words included in a plurality of texts;
a two-dimensional (2D) map generation unit that generates a plurality of pieces of 2D coordinate information by performing dimension compression on the plurality of feature vectors acquired by the feature vector acquisition unit and generates a 2D map in which positions corresponding to the plurality of feature vectors are visualized on a 2D plane based on the generated plurality of pieces of 2D coordinate information; and
a pathway generation unit that uses a similarity of the plurality of word feature vectors acquired by the feature vector acquisition unit or uses a position or a range designated in the 2D map generated by the 2D map generation unit to specify a plurality of molecules as words, and uses a knowledge database showing an intermolecular connection relationship for the specified plurality of molecules to generate a pathway representing an intermolecular interaction as a route map.

2. The information analysis apparatus according to claim 1, characterized in that
the feature vector acquisition unit acquires a disease feature vector which is a word feature vector specified for a name of a disease to be analyzed, and
the pathway generation unit specifies a plurality of molecules presumed to be related to the disease to be analyzed based on a similarity between the disease feature vector and a molecule feature vector which is a word feature vector specified for a name of a molecule, and generates the pathway using a knowledge database showing an intermolecular connection relationship for the specified plurality of molecules.

3. The information analysis apparatus according to claim 2, characterized in that
the pathway generation unit comprises
a related molecule estimation unit that inputs a disease feature vector acquired by the feature vector acquisition unit to a first trained model, thereby estimating a plurality of molecules related to the disease;
a molecular property estimation unit that inputs a disease feature vector specified for the disease to be analyzed and a molecule feature vector specified for the plurality of molecules estimated by the related molecule estimation unit to a second trained model, thereby estimating a probability that a molecule is causative or responsive as a property acting on the disease for each of the plurality of molecules; and
a route map generation unit that generates a pathway representing an intermolecular interaction as a route map in a manner that a causative molecule is on an upstream side and a responsive molecule is on an downstream side and that an intermolecular connection relationship shown by a known knowledge database is reflected for the plurality of molecules estimated by the related molecule estimation unit by using a property of a molecule estimated by the molecular property estimation unit and the knowledge database showing the intermolecular connection relationship.
wherein the first trained model is subjected to machine learning so as to output information about a molecule feature vector similar to the disease feature vector or a molecule corresponding to the molecule feature vector when the disease feature vector is input based on a similarity between the disease feature vector and the molecule feature vector.

4. The information analysis apparatus according to claim 3, characterized in that the second trained model is subjected to machine learning so as to output a probability that a property of a molecule is causative or responsive when the disease feature vector and the molecule feature vector are input using the disease feature vector, the molecule feature vector, and a data set of property information representing the property of the molecule acting on the disease as teacher data.

5. The information analysis apparatus according to claim 2, characterized in that
the feature vector acquisition unit acquires the disease feature vector, and acquires a molecule feature vector of a plurality of molecules presumed to be related to the disease to be analyzed based on a similarity between the acquired disease feature vector and the molecule feature vector, and
the 2D map generation unit generates a plurality of pieces of 2D coordinate information by performing dimension compression on the plurality of molecule feature vectors acquired by the feature vector acquisition unit, and generates a 2D map in which positions corresponding to the plurality of molecule feature vectors are visualized on a 2D plane based on the generated plurality of pieces of 2D coordinate information.

6. The information analysis apparatus according to claim 1, characterized in that
the feature vector acquisition unit acquires a molecule feature vector which is a word feature vector specified for a name of a molecule,
the 2D map generation unit generates a plurality of pieces of 2D coordinate information by performing dimension compression on a plurality of molecule feature vectors acquired by the feature vector acquisition unit, and generates a 2D map in which positions corresponding to the plurality of molecule feature vectors are visualized on a 2D plane based on the generated plurality of pieces of 2D coordinate information, and
the pathway generation unit specifies a plurality of molecules including one or more molecules corresponding to a position or a range designated in the 2D map generated by the 2D map generation unit and one or more other molecules whose connection relationship with the one or more molecules is shown by the knowledge database, and generates the pathway for the specified plurality of molecules.

7. The information analysis apparatus according to claim 1, characterized in that when an arbitrary route is designated in the pathway generated by the pathway generation unit, the 2D map generation unit displays a position of a molecule included in the route on the 2D map in manner that the position is distinguishable from a position of another molecule.

8. The information analysis apparatus according to claim 1, characterized in that when an arbitrary position or range is designated in the 2D map generated by the 2D map generation unit, the pathway generation unit displays a position of one or more molecules corresponding to the designated position or range on the pathway in manner that the position is distinguishable from a position of another molecule.

9. The information analysis apparatus according to claim 1, characterized in that
the feature vector acquisition unit acquires, as a plurality of word feature vectors specified for each of a plurality of words included in the plurality of texts, a plurality of feature vectors related to a name of at least one type among a plurality of molecule feature vectors which is word feature vectors specified for a name of a molecule, a plurality of disease feature vectors which is word feature vectors specified for a name of a disease, a plurality of drug feature vectors which is word feature vectors specified for a name of a drug, a plurality of compound feature vectors which is word feature vectors specified for a name of a compound, and a plurality of metabolite feature vectors which is word feature vectors specified for a name of a metabolite, and
the 2D map generation unit uses a plurality of feature vectors related to the name of the at least one type acquired by the feature vector acquisition unit to generate a 2D map related to the name of the at least one type.

10. The information analysis apparatus according to claim 9, characterized in that
the feature vector acquisition unit further acquires a plurality of text feature vectors specified for each of the plurality of texts, and
the 2D map generation unit generates a plurality of pieces of 2D coordinate information by performing dimension compression on the plurality of text feature vectors acquired by the feature vector acquisition unit, and further generates a 2D map in which positions corresponding to the plurality of text feature vectors are visualized on a 2D plane based on the generated plurality of pieces of 2D coordinate information.

11. The information analysis apparatus according to claim 10, characterized in that
the word feature vector is a vector representing a text to which a word included in the plurality of texts contributes and a degree at which the word contributes to the text, and
the text feature vector is a vector representing a word among a plurality of words included in the plurality of texts to which a text contributes and a degree at which the text contributes to the word.

12. The information analysis apparatus according to claim 9, characterized in that when an arbitrary position or range is designated on a 2D map of one type, the 2D map generation unit visualizes a position or a range on a 2D map of another type corresponding to the designated position or range, or displays one or more positions included in a range on the 2D map of the other type corresponding to the designated range in manner that the position is distinguishable from another position.

13. The information analysis apparatus according to claim 9, characterized in that when an arbitrary name related to a 2D map of one type is designated, the 2D map generation unit displays, on the 2D map of the one type, a position of a feature vector corresponding to the designated name in such a manner that the position is distinguishable from another position, and visualizes, in another type of 2D map, a position having the same 2D coordinates as 2D coordinates of the position which is displayed one the 2D map of the one type so as to be distinguishable from the other position.

14. The information analysis apparatus according to claim 9, characterized in that the pathway generation unit uses a disease 2D map which is a 2D map generated based on the disease feature vector and a molecule 2D map which is a 2D map generated based on the molecule feature vector to specify one or more molecules at a position or in a range on the molecule 2D map corresponding to the designated position or range in the disease 2D map, specifies a plurality of molecules including one or more other molecules whose connection relationship with the one or more molecules is shown by the knowledge database, and generates the pathway for the specified plurality of molecules.

15. An information analysis method characterized by comprising:
a step of using, by a 2D map generation unit of a computer, a plurality of word feature vectors specified for each of a plurality of words included in a plurality of texts to generate a 2D map in which positions corresponding to the plurality of word feature vectors are visualized on a 2D plane based on a plurality of pieces of 2D coordinate information obtained by performing dimension compression on each of the plurality of word feature vectors; and
a step of using, by a pathway generation unit of the computer, a similarity of the plurality of word feature vectors or using a position or a range designated in the 2D map to specify a plurality of molecules as words, and using a knowledge database showing an intermolecular connection relationship for the specified plurality of molecules to generate a pathway representing an intermolecular interaction as a route map.

16. The information analysis method according to claim 15, characterized in that the 2D map generation unit further uses a plurality of text feature vectors specified for each of the plurality of texts to further generate a 2D map in which positions corresponding to the plurality of text feature vectors are visualized on a 2D plane based on a plurality of pieces of 2D coordinate information obtained by performing dimension compression on each of the plurality of text feature vectors.

17. An information analysis program for causing a computer to function as:
2D map generation means for using a plurality of feature vectors including a plurality of word feature vectors specified for each of a plurality of words included in a plurality of texts to generate a plurality of pieces of 2D coordinate information by performing dimension compression on the plurality of feature vectors, and generating a 2D map in which positions corresponding to the plurality of feature vectors are visualized on a 2D plane based on the generated plurality of pieces of 2D coordinate information; and
pathway generation means for using a similarity of the plurality of word feature vectors or using a position or a range designated in the 2D map generated by the 2D map generation means to specify a plurality of molecules as words, and using a knowledge database showing an intermolecular connection relationship for the specified plurality of molecules to generate a pathway representing an intermolecular interaction as a route map.

18. The information analysis apparatus according to claim 3, characterized in that when an arbitrary route is designated in the pathway generated by the pathway generation unit, the 2D map generation unit displays a position of a molecule included in the route on the 2D map in manner that the position is distinguishable from a position of another molecule.

19. The information analysis apparatus according to claim 3, characterized in that when an arbitrary position or range is designated in the 2D map generated by the 2D map generation unit, the pathway generation unit displays a position of one or more molecules corresponding to the designated position or range on the pathway in manner that the position is distinguishable from a position of another molecule.
